(19) United States
(12) Patent Application Publication
(10) Pub. No.: US 2016/0242476 A1
(43) Pub. Date: Aug. 25, 2016

(54) HIP PROTECTIVE DISPOSABLE UNDERGARMENT

(71) Applicant: ROBERT NUTTER, RENO, NV (US)

(72) Inventor: ROBERT NUTTER, RENO, NV (US)

(21) Appl. No.: 14/626,386

(22) Filed: Feb. 19, 2015

(51) Int. Cl. A41D 13/015; A61F 7/02; A41D 13/05

(52) U.S. Cl. CPC A41D 13/0153; A41D 13/0506; A41D 13/0531; A61F 7/02; A61F 2007/004

(57) ABSTRACT

A hip protective, disposable undergarment is described. The hip protective, disposable undergarment includes a brief having a body between a first end and a second end. The first end includes a waist opening, and the second end includes two leg openings. A first pocket is included in the body, and a first hip protective insert is removably attached to the first pocket of the body. The first hip protective insert is configured to be disposed over a first trochanteric region of the wearer when the undergarment is worn. A second pocket is also included in the body and a second hip protective insert is removably attached to the second pocket of the body. The second hip protective insert is disposed over a second trochanteric region of the wearer when the undergarment is worn.

### CROSS REFERENCE

The present patent application claims the benefit of provisional patent application No. 62/942,563 filed Feb. 20, 2014.

### FIELD OF THE INVENTION

The present invention is relates to a hip protective, disposable undergarment. More specifically, the invention relates to a hip protective, disposable undergarment for incontinent persons.

### BACKGROUND

Hip injuries are typically associated with the elderly. The elderly face significant risk of long term functional impairment, nursing home admission, and mortality as a result of hip fractures following a fall from a standing or a near standing position. More than 95% of hip fractures in the elderly are caused by a sideways fall onto the hip. Such falls result in a direct impact on the greater trochanter region of the proximal femur. Such falls are high-impact injuries often creating an impact force which exceeds the breaking strength of the bone.

The number of hip fractures is expected to rise because of the rising population of elderly. There are significant compliance issues with getting elderly patients to use hip protectors. Compliance issues arise as a result of difficulty of putting on hip protectors. Also, hip protectors are reported to be uncomfortable and not aesthetically pleasing due to thickness and shape of the hip protective device. Other factors leading to reduced compliance include dementia and a perceived lack of personal risk of a fall. Yet another reason for noncompliance is skin irritation stemming from the hip protector.

Hip protectors are associated with a reduction in hip fractures in the elderly in institutional settings, although not in residential settings. It is believed that this reduction in efficiency stems from a reduction in patient compliance in residential settings. Most protective undergarments reduce the force of the impact of a fall by cushioning the trochanter with a pad or pads, which absorb the force of an impact, dissipating the impact force over a larger surface area than the trochanter; thereby, preventing a fracture from a fall.

Other protective undergarments reduce the force of the impact from a fall by having an impact-deflecting portion, such as a hole or a concavity, which deflects the impact force from the trochanter region. The impact force is also dissipated across a larger surface area to prevent bone fracture.

However, there are many disadvantages for hip protective undergarments having an impact-deflecting portion that must be worn over an incontinence undergarment. Firstly, the hip protective undergarments require more work to put on and take off because there are two undergarments. Secondly, the two undergarments are not aesthetically pleasing. Thirdly, if a garment is not aesthetically pleasing, the user will not wear the undergarment, and fractures from falls are thereby not prevented. Fourthly, the hip protective undergarments do not protect the rear pelvis.

A washable undergarment having hip protective pads has the additional disadvantage of causing bacterial cross-contamination. Such cross-contamination occurs when a soiled diaper is removed, replaced, and the hip-protective undergarment is then re-used on the patient. Even if the hip protective undergarment is removed and sent to a laundry, it passes through staff work areas, and may be a source of cross-contamination for bacteria.

Typically, a washable undergarment having hip protective pads has the further disadvantage of requiring “turn-around” time when soiled undergarments are sent to the laundry for washing. Such undergarments may also become lost or stolen while in the laundry or another user's undergarment may be sent back to the patient.

In addition, present day hip protective undergarments are difficult to remove from a patient. It generally takes two staff persons to remove a hip protective undergarment to avoid injury to the patient. This is often painful and uncomfortable for the patient, and time consuming for the hospital.

Some adult incontinence devices integrate a cushion into an adult diaper. However, disposable adult diapers with a cushion have the disadvantage of being bulky and thick, which makes them unpleasing for patients—resulting in wearer noncompliance. Another disadvantage with incontinence devices with cushions is that they are expensive to manufacture, ship, and store.

### SUMMARY

A hip protective, disposable undergarment is described. The illustrative hip protective, disposable undergarment includes a brief having a body between a first end and a second end. The first end includes a waist opening, and the second end includes two leg openings. A first pocket is included in the body and a first hip protective insert is removably attached to the first pocket of the body. The first hip protective insert is configured to be disposed over a first trochanteric region, e.g. left side of hip. A second pocket is also included in the body, and a second hip protective insert is removably attached to the second pocket of the body. The second hip protective insert is configured to be disposed over a second trochanteric region, e.g. right side of the hip.

The illustrative embodiment may also include a third pocket configured to receive a spine protective insert is removably attached to the third pocket of the body, the spine protective insert is disposed over the coccyx of the wearer when the undergarment is worn. In one embodiment of the hip protective undergarment, the body has a tubular shape. In another embodiment of the hip protective, disposable undergarment, the body has an H-shape.

### FIGURES

The present invention will be more fully understood by reference to the following drawings which are for illustrative, not limiting, purposes.

FIG. 1A shows a front view of a human skeleton.

FIG. 1B shows a back view of a human skeleton.

FIG. 1C shows a human pelvis.

FIG. 1D shows a human femur.

FIG. 1E shows a femoral neck fracture of a human femur.

FIG. 1F shows an intertrochanteric fracture of a human figure.

FIG. 2 shows a front view of the illustrative hip protective undergarment with the first and second pockets disposed over the greater trochanter region of the proximal femur of a wearer.

FIG. 3A shows a front view of the illustrative hip protective undergarment with the hip protective inserts not disposed within the first and second pockets.

FIG. 3B shows a back view of the illustrative hip protective undergarment with the spine protective insert not disposed within the third pocket.

FIG. 4A shows a front view of the illustrative hip protective undergarment with the hip protective inserts disposed within the first and second pockets.

FIG. 4B shows a back view of the illustrative hip protective undergarment with the spine protective insert disposed within the third pocket.

FIG. 5A shows a side of the illustrative hip protective undergarment having an H-shaped body which contacts the patient.

FIG. 5B shows the side of the illustrative hip protective undergarment having an H-shaped body which is opposite the patient.

### DESCRIPTION

Persons of ordinary skill in the art will realize that the following description is illustrative and not in any way limiting. Other embodiments of the claimed subject matter will readily suggest themselves to such skilled persons having the benefit of this disclosure. It shall be appreciated by those of ordinary skill in the art that the systems and apparatus described hereinafter may vary as to configuration and as to details. Additionally, the methods may vary as to details, order of the actions, or other variations without departing from the illustrative methods disclosed herein.

The various hip protective, disposable undergarment embodiments presented herein prevent infection and protect the wearer from a hip injury. Additionally, the disposable hip protective garment is transportable and can be easily stored. The various hip protective, disposable undergarment embodiments presented herein may be removed and replaced by the wearer, or by the wearer with the assistance of one person only; thereby, reducing staff labor costs in an institutional setting such as a nursing home.

The illustrative hip protective undergarment may be a disposable incontinence brief, affording an aesthetically pleasing alternative to wearing a hip protective undergarment over a disposable incontinence brief; thus, overcoming user noncompliance due to the thickness of the garment. In one illustrative embodiment, the hip protective undergarment has a spine protective insert which protects both the trochanteric region and the rear pelvis from fracture due to the impact force of a fall.

The number of hip fractures is expected to increase as the population of elderly persons rises. Ninety percent of hip fractures occur in patients who are 60 years of age or older. The incidence of hip fractures doubles every decade after the age of 50, and about 80% of hip fractures occur in females. The Asian and Caucasian population is more highly affected due to higher levels of osteoporosis, which causes bones to become thinner as a result of loss of calcium. Women lose about 30-50% of their bone density as they age, and this bone loss speeds up after menopause.

Disposable undergarments are used for the elderly and others who suffer from incontinence. When the undergarment is soiled, it is removed and discarded, and the hip protector undergarment is then either sent to the laundry, or just put back on the wearer. However, this produces conditions which are ripe for bacterial contamination.

Bacterial transmission of antibiotic associated diarrhea such as Clostridium difficile (C. difficile) is a serious health concern in institutional settings such as hospitals, long term care facilities, and nursing homes. C. difficile causes watery diarrhea and mild abdominal pain and tenderness in most people. However, C. difficile can cause severe diarrhea, severe abdominal pain and cramping, fever, nausea, dehydration, weight loss, pseudomembranous colitis, kidney failure and death. Elderly persons and those with compromised immune systems are at a higher risk for C. difficile infection.

C. difficile spores are not effectively killed by alcohol-based hand rubs. Removing C. difficile spores from soiled clothing is accomplished with a washing machine. However, moving soiled clothing through a hospital from a patient's room to the laundry results in bacterial transmission from the soiled clothing to the patient's clothing or skin.

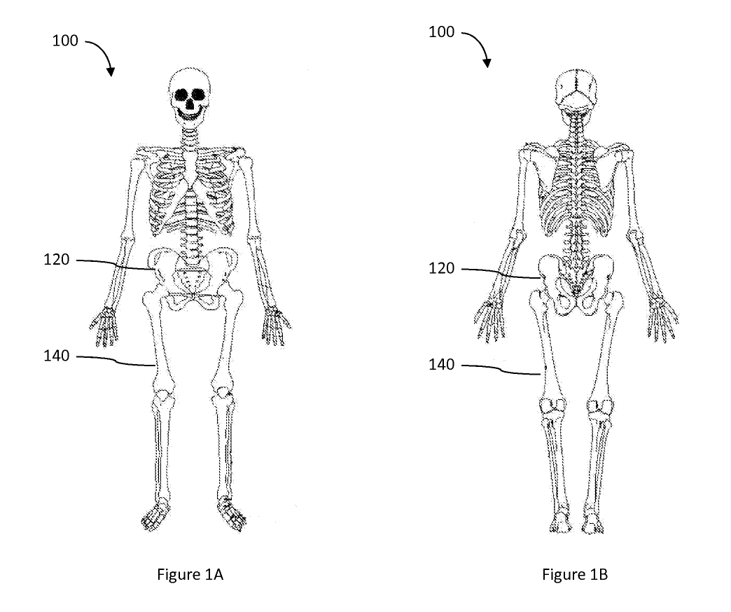

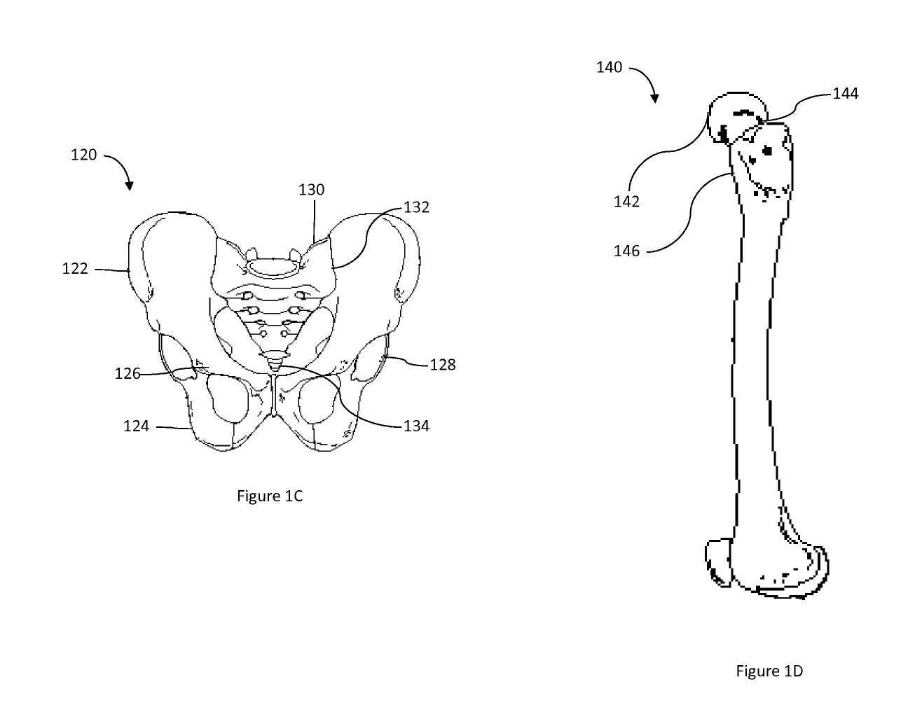

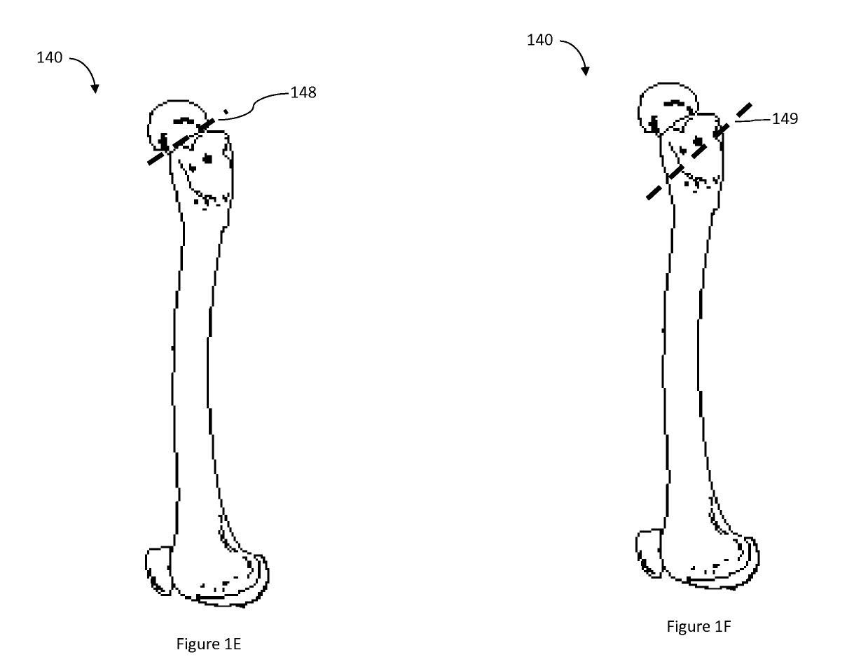

Referring now to FIG. 1A, there is shown a front view of a human skeleton 100. The pelvis 120 includes a ring of bones in the lower torso of the human body, connecting to the large upper leg bone known as the femur 140. FIG. 1B shows a back view of the human skeleton 100.

Referring now to FIG. 1C, there is shown an illustrative pelvis 120 of the human skeleton 100. The pelvis 120 is composed of three fused hip bones, the ilium 122, the ischium 124, and the pubis 126. There is a cup-like depression in the pelvis 120 called the acetabulum 128. The pelvis 120 is joined to the sacrum 130 at the sacroiliac joints 132. The sacrum 130 is the large, triangular bone at the base of the spine which joins to the coccyx 134, otherwise known as the tailbone, at its lowest part.

The coccyx 134 is the lowest part of the vertebral column. The coccyx 134 moves forward when a person is sitting, acting as a shock absorber. When a person sits down with too much force, such as a fall into a sitting position, this may cause the coccyx 134 to be fractured or dislocated. The rear pelvic region as presented herein includes the coccyx 134.

Referring now to FIG. 1D, there is shown an illustrative view of a femur 140 of the human skeleton 100. The upper portion of the femur 140 is composed of the greater trochanter 142, the femoral neck 144 and the lesser trochanter 146. The hip joint is a ball and socket joint where the ball-like greater trochanter 142 of the femur 140 fits into the cup-like acetabulum 128.

Together, the greater trochanter 142, the femoral neck 144, and the lesser trochanter 146 are called the trochanteric region. The trochanteric region is less dense than the rest of the femur.

For purposes of this patent, a “first trochanteric region” includes the greater trochanter 142, the femoral neck 144, and the lesser trochanter 146 of the left side of patient. A “second trochanteric region” includes the greater trochanter 142, the femoral neck 144, and the lesser trochanter 146 of the right side of a patient. By way of example and not of limitation, the first trochanteric region may refer to a patient's “left hip” and the second trochanteric region may refer to a patient's “right hip.”

Referring now to FIG. 1E, there is shown a picture of the femur 140 of the human skeleton 100. Femoral neck fractures occur in approximately the area shown by the dashed line 148. Femoral neck fractures occur one or two inches from the hip joint.

Referring now to FIG. 1F, there is shown a picture of the femur 140 of the human skeleton 100. Intertrochanteric hip fractures occur in approximately the area shown by the dashed line 149.

The majority of fractures in the elderly population are femoral neck fractures and intertrochanteric hip fractures. Hip fractures result in loss of mobility and independent living in the elderly, as well as depression. About 20% of persons who suffer a hip fracture die within a year of the fracture. Hip fractures are high-impact injuries often creating an impact force which exceeds the breaking strength of the bone.

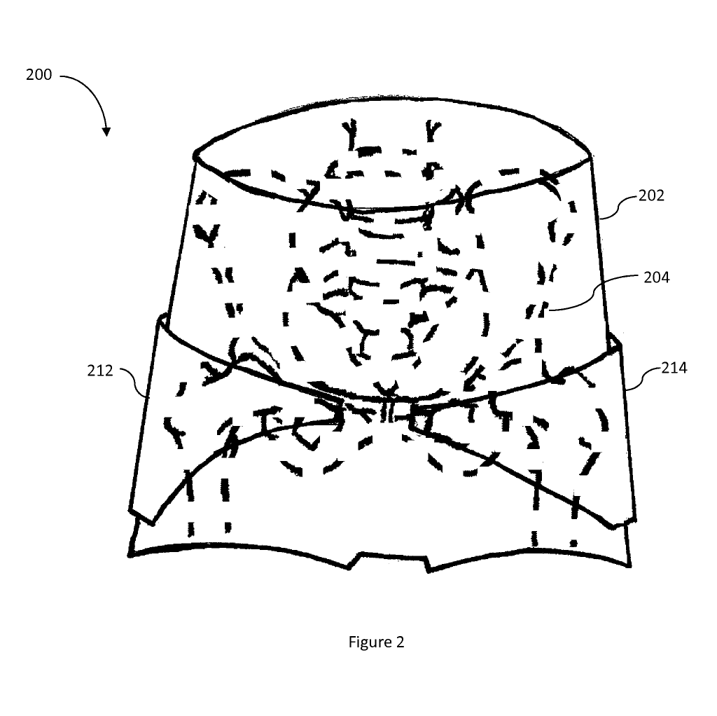

Referring now to FIG. 2, there is shown the illustrative hip protective, disposable undergarment 200 that includes a plurality of pockets. In the illustrative undergarment 200, the body 202 includes a tubular body in a pull-up type of adult incontinence undergarment. The illustrative wearer's hip bones 204 are presented with dashed lines.

The undergarment 200 includes a plurality of pockets 212 and 214. Each pocket is a small bag-like attachment sewn into the hip protective, disposable undergarment and used to carry protective inserts. The first illustrative pocket 212 is disposed over a first trochanteric region of the wearer. The second illustrative pocket 214 is disposed over a second trochanteric region of the wearer.

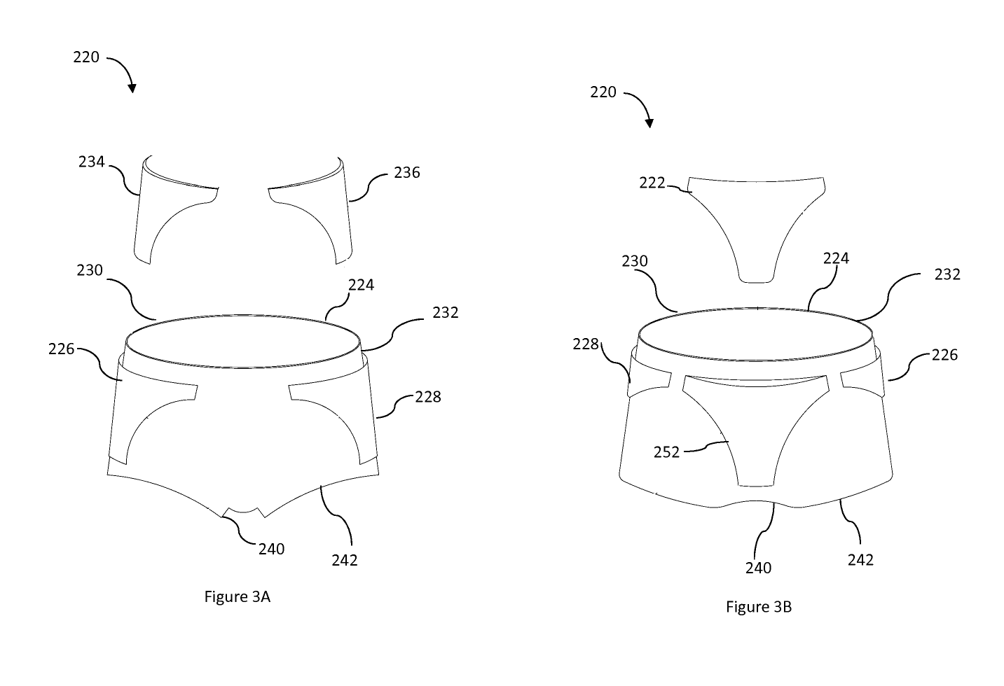

Referring now to FIG. 3B, there is shown a front view of another illustrative hip protective, disposable undergarment 220 that includes a spine protective insert 222. The hip protective, disposable undergarment 220 includes a brief having a body 224 between a first end 230 and a second end 240. The first end 230 includes a waist opening 232, and the second end 240 includes two leg openings 242.

The body 224 also includes a first pocket 226 and a second pocket 228. A first hip protective insert 234 is removably attached or received by the first pocket 226 of the body 224. Referring now to FIG. 3A, the first hip protective insert 234 is disposed over a first trochanteric region of the wearer when the hip protective undergarment 220 is worn by the user.

The body 224 further includes a second pocket 228 configured to receive a second hip protective insert 236. The second hip protective insert 236 is disposed or located over a second trochanteric region of the wearer when the hip protective undergarment 220 is worn by the user.

The illustrative hip protective inserts presented herein may be roughly triangular in shape. More generally, the hip protective inserts may be of any shape or size which would adequately protect the trochanteric region and the coccyx from fracture during falls. For example, the hip protective inserts may have tapered edges, in which the insert is thickest at the center and then tapers to thin edges to avoid a bulky appearance, increasing aesthetic appeal. By tapering the edges of the insert, the insert does not stand out of the user's clothing at odd angles, thereby enhancing the aesthetic appeal from the hip protective undergarment and increasing compliance.

In yet another illustrative embodiment, the hip protective inserts are not disposed within pockets. Instead the hip protective inserts are attached to the exterior of the undergarment with an illustrative adhesive or other such fastener, which does not require using pockets for holding the inserts in place.

Referring back to FIG. 3B, there is shown a back view of an illustrative hip protective undergarment. The tubular body 224 further includes a third pocket 252. A spine protective insert 222 is removably attached or received by the third pocket 252 of the tubular body 224. The spine protective insert 222 is configured to be located or disposed over the coccyx 134 of the wearer to protect the coccyx 134 from fractures due to falls. In operation, the spine protective insert 222 absorbs the impact force of a fall into a seated position, spreading the force over a larger surface area, thereby preventing fracture of the coccyx 134.

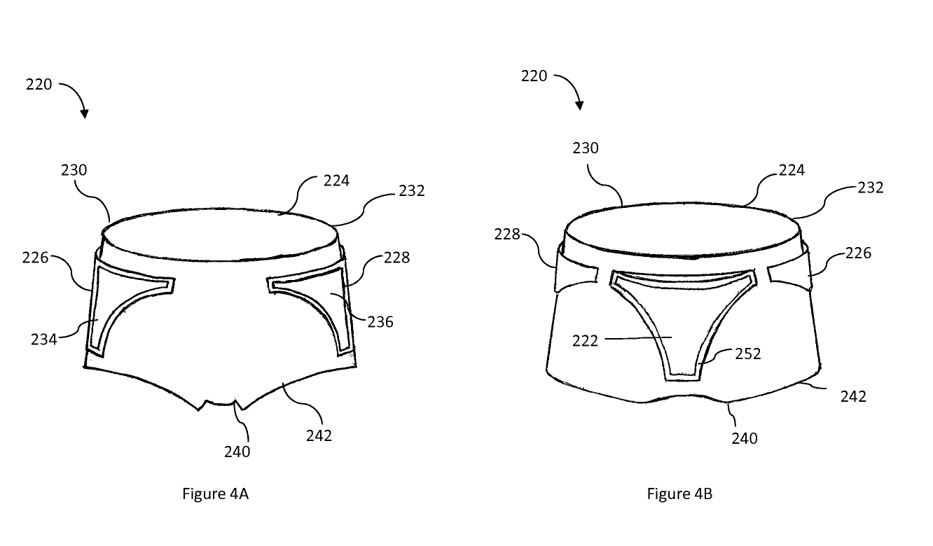

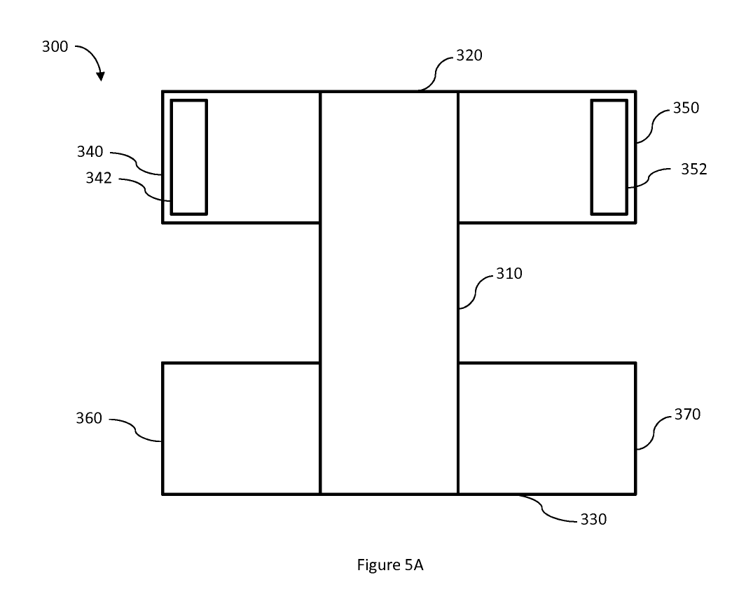

Referring now to FIG. 4A, there is shown a front view of the illustrative hip protective, disposable undergarment 220 presented above in FIG. 3A, in which the inserts have been received by the pockets of the undergarment. More specifically, the body 224 of the hip protective, disposable undergarment 220 is shown with the first hip protective insert 234 disposed inside of a first pocket 226. The tubular body 224 is further shown with the second hip protective insert 236 disposed inside of the second pocket 228 of the tubular body 224. The first pocket 226 and the second pocket 228 of the tubular body 224 may be closed and thereby fix or retain the first hip protective insert 234 and the second hip protective insert 236 within the corresponding pockets. Referring now to FIG. 4B, there is shown a back view of an illustrative hip protective undergarment, in which the spine protective insert 222 is shown disposed inside of the third pocket 252.

There may other alternative embodiments configured to keep the hip protective inserts from moving. For example, a hook and loop type fastener, buttons, adhesive strips, Velcro, or other such fasteners or materials may be used to keep the hip protective inserts in place. Thus, although a pocket is presented in the illustrative embodiment, the hip protective inserts may also be placed directly on the exterior surface of the disposable undergarment by modifying the undergarment. For example, the undergarment and inserts may be removably coupled to one another by utilizing one or more fasteners, modifying the undergarment, utilizing a material that enables the inserts to be removably coupled to the undergarment, or any combination thereof.

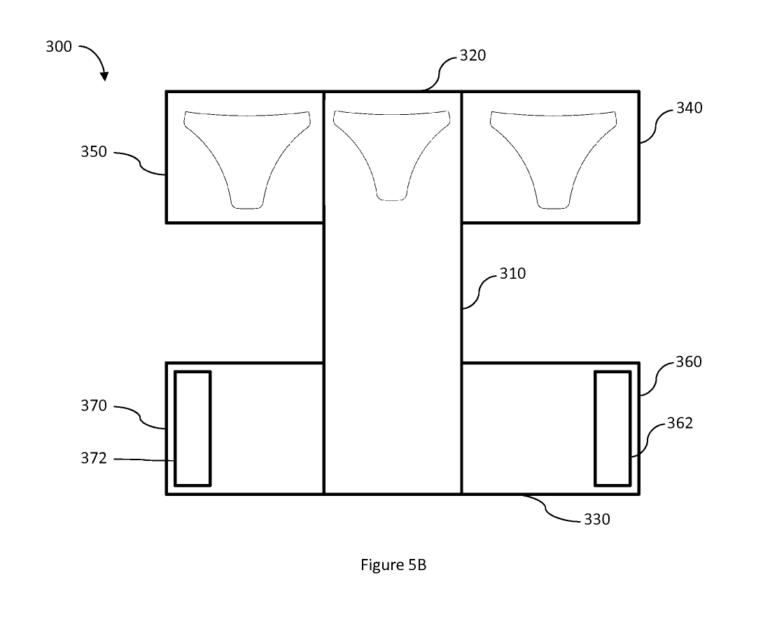

Referring now to FIG. 5A, there is shown the face of an H-shaped diaper that contacts a patient. Diapers which are of the “pull-on” type can be very difficult to get on and off a bedridden elderly patient. By using an H-shaped diaper for bedridden patients, pain and skin irritation are avoided. Skin irritation can result in bed sores which lead to infection and untimely death. By using a diaper having an H-shaped body, the caregiver can also smooth the diaper over the patient's skin, resulting in no wrinkles in the diaper. Wrinkles in an adult diaper can cause bedsores, and infection.

The illustrative hip protective, disposable undergarment having an H-shaped body is composed of a rectangular portion 310 having a first end 320 and a second end 330. The hip protective, disposable undergarment having an H-shaped body also includes a first wing 340, a second wing 350, a third wing 360, and a fourth wing 370. The rectangular portion 310 and the wings 340, 350, 360 and 370 form an H-shape. When worn by the wearer, the rectangular portion 310 is placed under a patient's pelvis. The first wing 340 and second wing 350 are positioned on the patient's posterior, and the third wing 360 and fourth wing 370 are placed over the patient's groin. The first wing 340 is placed over, and removably attached to the third wing 360. The second wing 350 is placed over and removably attached to the fourth wing 370.

The wings may be attached with a hook and loop fastener, such as Velcro, or other fastening means, such as adhesive, so that the diaper can be removed without pulling down. In one illustrative embodiment, the first wing 340 includes a first hook fastener 342, and the second wing 350 includes a second hook fastener 352.

Referring now to FIG. 5B, there is shown the outside of the illustrative hip protective, disposable undergarment 300 having an H-shaped body. The outside of the illustrative hip protective, disposable undergarment 300 is opposite the patient's skin. The outside of the illustrative hip protective, disposable undergarment 300 having an H-shaped body includes the rectangular portion 310, and has the first end 320 and the second end 330. The hip protective, disposable undergarment having an H-shaped body 300 also includes the first wing 340, the second wing 350, the third wing 360 and the fourth wing 370. Third wing 360 includes a first loop fastener 362, and fourth wing 370 includes a second loop fastener 372. When the first wing 340 is placed over the third wing 360, the first hook fastener 342 removably attaches to the first loop fastener 362. When the second wing 350 is placed over the fourth wing 370, the second hook fastener 352 removably attaches to second hook fastener 372.

The inserts described herein may be used for cryotherapy. Cryotherapy is a safe and effective way to relieve pain following abdominal surgery. Cryotherapy tends to reduce swelling in a wound. In operation, the illustrative inserts presented herein may be frozen or chilled. The inserts may then be inserted into the pockets after surgery with or without the hip protective inserts. Smooth sided inserts which have been frozen or chilled present greater patient comfort over ice packs which are taped or tied to a surgical site.

The inserts described herein may also be used for thermotherapy, which is known to assist with healing. Thermotherapy is the application of heat to the body to cause vasodilation, which allows more blood flow, increasing the amount of oxygen and nutrients to cells, and allowing waste products such as carbon dioxide and metabolic waste to flow out of cells. Thermotherapy can be applied through moist heat transfer, such as through a hot bath or hot water, or the transfer of heat through air, such as in a sauna. Other methods of heat transfer are ultrasound, heating pad, or far or near infrared radiation. Further, the use of thermotherapy in conjunction with the application of a cryotherapy may also be used to reduce swelling. By way of example and not of limitation, the thermotherapy inserts may be electrically coupled to a power source so that the inserts may be electrically heated.

Additionally, the illustrative protective inserts described above may also include an impact-absorbing pad. The illustrative inserts may each be shaped differently or may be customized according to the size and shape of the patient. The protective inserts may include an impact-absorbing pad and an impact-deflecting portion comprising a hole or a concavity.

The illustrative hip protective inserts may also be composed of a silicone material or other such thermoplastic material. A thermoplastic, or thermosoftening plastic, is a polymer which becomes moldable above a certain temperature, and returns to a solid state upon cooling. One such illustrative thermoplastic is silicone.

Silicone hip protectors prevent hip fractures by acting as a shock absorber during falls as compared to falls without the use of silicone hip protectors. Silicone is compressible without absorbing water or other fluids that the pad is exposed to, and is stable over a wide range of temperatures. Additionally, medical grade silicone is hypo-allergenic, offering comfort, cushioning, and shock absorption.

Other types of shock absorbers include expanded rubber, also called foam rubber or EVA. EVA is impervious to liquids, and is used as a shock absorber in sports shoes and in athletic padding. Yet another shock absorber is sorbothane, used for shock absorption in heel pads.

The illustrative hip protective inserts may also include a hard plastic outer shell with soft shock-absorbing padding under the outer shell. The use of a hard outer shell hip protective pad reduces impact force of a fall to surrounding soft tissues, and thereby avoids a fracture of the trochanter.

The hip protective, disposable undergarment presented herein supports less painful repositioning of a patient during changing, because there is less soiled clothing and bedding which needs replacing. Since a disposable undergarment is used, there is no need to wait for the undergarment to be returned from the laundry. Further, there are no undergarments which will be accidentally placed on the wrong patient.

Additionally, the patient may have more than one set of reusable hip and spine protective inserts, so that when one set is being disinfected or washed, the other set(s) is available for reuse. The hip and spine protective inserts are small enough that these may easily be stored near a patient's bedside.

By supplying a hip protective insert which can be disinfected and which is reusable, the hip protective undergarment provides a cost-effective alternative to the high shipping, storage, and production costs of prior art adult incontinence briefs having a cushioning means.

Additionally, the hip protective inserts may be composed of a substance that can be washed in a clothes washer and dried in a dryer. Thus, in conjunction with a disposable undergarment, the hip protective inserts provides hip protection and minimizes the patient's exposure to bacterial infection such as C. difficile.

Furthermore, the hip protective inserts may be molded to the particular patient and are thereby more comfortable to wear, more aesthetically appealing, and prevent skin irritation.

It is to be understood that the detailed description of illustrative embodiments are provided for illustrative purposes. The scope of the claims is not limited to these details, and uses can differ from those just described, or be expanded on or implemented using technologies not yet commercially viable, and yet still be within the inventive concepts of the present disclosure. The scope of the invention is determined by the following claims and their legal equivalents.

What is claimed is:

1. A hip protective, disposable undergarment comprising:
a disposable incontinence undergarment having a body between a first end and a second end, wherein the first end includes a waist opening, and the second end includes two leg openings;
a first hip protective insert that is removably attached to the body, wherein the first hip protective insert is configured to be disposed over a first trochanteric region;
a second hip protective insert that is removably attached to the body, wherein the second hip protective insert is configured to be disposed over a second trochanteric region; and
a spine protective insert that is removably attached to the body, wherein the spine protective insert is disposed over a rear pelvic region.

2. The hip protective, disposable undergarment of claim 1 wherein the body has a tubular shape.

3. The hip protective, disposable undergarment of claim 11 wherein the body has an H-shape, including a rectangular portion, a first wing, a second wing, a third wing and a fourth wing.

4. The hip protective undergarment of claim 1 wherein the first hip protective insert is disposed within a first pocket, the second hip protective insert is disposed within a second pocket, and the spine protective insert is disposed inside of a third pocket.

5. The hip protective undergarment of claim 2 wherein the first hip protective insert, the second hip protective insert, and the spine protective insert include silicone.

6. The hip protective undergarment of claim 2 wherein the first hip protective insert, the second hip protective insert, and the spine protective insert include a thermoplastic polymer.

7. The hip protective undergarment of claim 1 wherein the first hip protective insert, the second hip protective insert, and the spine protective insert include an outer hard shell and an inner shock-absorbing soft shell.

8. The hip protective undergarment of claim 1 wherein the first hip protective insert, the second hip protective insert, and the spine protective insert are configured to be heated during thermotherapy and cooled during cryotherapy.

9. The hip protective undergarment of claim 1 wherein the first hip protective insert, the second hip protective insert, and the spine protective insert are configured to be disinfected by washing with soap and water.

10. The hip protective undergarment of claim 1 wherein the first hip protective insert, the second hip protective insert, and the spine protective insert are configured to be secured with a hook and loop fastener.

11. A hip protective undergarment comprising:
a disposable incontinence undergarment having a body between a first end and a second end, wherein the first end includes a waist opening, and the second end includes two leg openings;
a first hip protective insert that is removably attached to the body, wherein the first hip protective insert is configured to be disposed over a first trochanteric region; and
a second hip protective insert that is removably attached to the body, wherein the second hip protective insert is configured to be disposed over a second trochanteric region.

12. The hip protective, disposable undergarment of claim 11 wherein the body has a tubular shape.

13. The hip protective, disposable undergarment of claim 11 wherein the body has an H-shape, including a rectangular portion, a first wing, a second wing, a third wing and a fourth wing.

14. The hip protective undergarment of claim 11 wherein the first hip protective insert is disposed within a first pocket and the second hip protective insert is disposed within a second pocket.

15. The hip protective undergarment of claim 11 wherein the first hip protective insert and the second hip protective insert include silicone.

16. The hip protective undergarment of claim 11 wherein the first hip protective insert and the second hip protective insert include a thermoplastic polymer.

17. The hip protective undergarment of claim 11 wherein the first hip protective insert and the second hip protective insert include an outer hard shell and an inner shock-absorbing soft shell.

18. The hip protective undergarment of claim 11 wherein the first hip protective insert and the second hip protective insert are configured to be heated during thermotherapy and cooled during cryotherapy.

19. The hip protective undergarment of claim 11 wherein the first hip protective insert and the second hip protective insert are configured to be disinfected with soap and water.

20. The hip protective undergarment of claim 11 wherein the first hip protective insert and the second hip protective insert are configured to be secured with a hook and loop fastener.
